(19) United States
(12) Patent Application Publication
(10) Pub. No.: US 2004/0236127 A1
(43) Pub. Date: Nov. 25, 2004

(54) Method for hardening unsaturated fatty substances

(76) Inventors: Gerrit Pelzer, Duesseldorf (DE); Lars Zander, Duesseldorf (DE); Ulrich Ruigk, Krefeld (DE); Lothar Friesenhagen, Duesseldorf (DE); Lothar Friesenhagen, Duesseldorf (DE); Bernhard Gutsche, Hilden (DE); Norbert Klein, Mettmann (DE); Ingomar Mrozek, Duesseldorf (DE); Egbert Gritz, Duesseldorf (DE); Horst Josten, Duesseldorf (DE); Wilhelm Kuhr, Neuss (DE)

Correspondence Address: COGNIS CORPORATION, PATENT DEPARTMENT, 300 BROOKSIDE AVENUE, AMBLER, PA, 19002, US

(21) Appl. No.: 10/488,306

(22) PCT Filed: Aug. 21, 2002
(86) PCT No.: PCT/EP02/09321

(30) Foreign Application Priority Data
Aug. 30, 2001 (DE) 101 42 306.3

(51) Int. Cl. C07C 51/36

(52) U.S. Cl. 554/141

(57) ABSTRACT

Unsaturated fatty compounds are hydrogenated at a hydrogen pressure of from 100 to 300 bar in the presence of transition metal catalyst to form a hydrogenation product having an iodine value of less than 2.

### FIELD OF THE INVENTION

This invention relates generally to oleochemical raw materials and, more particularly, to an industrial process for hydrogenating unsaturated compounds.

### PRIOR ART

Oleochemical raw materials from natural fats and oils to the first refinement products, namely fatty acids, fatty acid esters and fatty alcohols, are mixtures of unsaturated and saturated homologs, the degree of unsaturation—as expressed by the so-called iodine value—being largely determined by the raw material used. Besides unsaturated natural materials, however, synthetic materials obtained by fermentation of suitable paraffins are now also acquiring significance. Particular mention is made of long-chain dicarboxylic acids obtained by bio-oxidation of paraffins or monocarboxylic acids in the presence of Candida tropicalis [cf. EP 0229252 A1 (Henkel)].

Although the unsaturation of a compound is an extremely valuable property for many applications, for example in the cosmetics field, it is appropriate for other applications to saturate, i.e. “hydrogenate”, the double bonds present either completely or at least partly without aversely affecting other functions in the molecule. An important example of this is provided by the margarine industry although, judging by the annual production figures, hydrogenation is also extremely important in the field of chemical raw materials. Hydrogenation is normally carried out in the presence of transition metals, such as nickel or palladium for example, in order to prevent the carboxyl function from being hydrogenated during saturation of the double bonds in cases where unsaturated esters or acids are used. Typical conditions are temperatures in the range from 180 to 280° C. and hydrogen pressures of up to 25 bar [cf. The Basics of Industrial Oleochemistry, G. Dieckelmann (Ed.), 1988, pp. 75-81].

Although, in recent decades, great efforts have been made to improve existing hydrogenation processes, the results obtained are, even today, still in need of improvement in some respects. For example, in 1993, applicants developed a new transition metal catalyst for the hydrogenation of fats which was distinguished from the prior art by the fact that lower iodine values were obtained for a longer catalyst life. However, in this and other processes, the throughput of the production plant, as characterized by a liquid hourly space velocity (LHSV) of well below 0.5 h⁻¹, is still far removed from what would be desirable from the production perspective, for example in order to be able to construct smaller plants with the same capacity, but with less capital investment.

Accordingly, the problem addressed by the present invention was to provide an improved process for hydrogenating unsaturated fatty compounds, more particularly unsaturated carboxylic acids and, above all, unsaturated dicarboxylic acids, with which iodine values below 2, preferably below 1 and more particularly below 0.5 could be achieved; at the same time, the reaction rate would be distinctly increased in relation to the prior art.

### DESCRIPTION OF THE INVENTION

The present invention relates to a process for hydrogenating unsaturated fatty compounds in the presence of transition metal catalysts which is characterized in that saturation of the double bonds is carried out under pressures of 100 to 300 bar, preferably 150 to 280 bar and more particularly 200 to 250 bar.

It is known from numerous experiments with discontinuous hydrogenation that, under pressures of 15 to 20 bar, the hydrogen passes directly into the liquid phase, so that any further increase in the reaction pressure would be unable to produce a significant reduction in the hydrogenation time, but would increase the costs of the process in regard to the design of the reactor and the expansion of the reaction products from a relatively high level. For this reason, low-pressure processes are exclusively used on an industrial scale. However, applicants have found that, contrary to all expectations, an increase in the pressure to at least 100 bar, preferably to 150 bar and more particularly to 200 bar leads to a significant increase in the reaction rate, in some cases by a factor of up to 10. As explained, this cannot be accounted for by faster dissolution and diffusion of the hydrogen; instead, it is assumed that the catalyst is permanently regenerated in situ, especially since there was no sign of exhaustion in the tests carried out to date. Accordingly, the invention provides a hydrogenation process for virtually any unsaturated fatty compounds which, although involving higher investment costs in regard to the design of the pressure vessel, is able to use much smaller reactors by virtue of the far higher conversion level.

Unsaturated Fatty Compounds

The choice of the unsaturated fatty compounds used as educts for the hydrogenation is basically not critical. Typically, unsaturated carboxylic acids, unsaturated carboxylic acid alkyl esters and unsaturated carboxylic acid polyol esters may be used for this purpose. The unsaturated carboxylic acids preferably correspond to formula (I):

R¹—[A¹]—COOH (I)

in which A¹ is a mono- or polyunsaturated, linear or branched hydrocarbon radical containing 5 to 21 carbon atoms and R¹ is hydrogen or a carboxyl group, i.e. the carboxylic acids may be both mono- and dicarboxylic acids, dicarboxylic acids being preferred insofar as they produce the clearest increase in the reaction rate. Typical examples of suitable monocarboxylic acids are fatty acids containing 6 to 22 and preferably 16 to 22 carbon atoms, such as palmitoleic acid, oleic acid, elaidic acid, petroselic acid, linoleic acid, linolenic acid, gadoleic acid and behenic acid and technical mixtures thereof. Also suitable are branched unsaturated fatty acids which accumulate as monomer fraction in the dimerization of unsaturated monocarboxylic acids, especially oleic acid. Suitable dicarboxylic acids are those containing 3 to 22 carbon atoms such as, for example, maleic acid and fumaric acid and, more particularly, the unsaturated dicarboxylic acids which are obtained by enzymatic carboxylation of the corresponding paraffins. Unsaturated C₁₈ dicarboxylic acid in particular is mentioned in this regard. Finally, other suitable starting materials are the branched, optionally naphthenic di- and tricarboxylic acids which are formed in the oligomerization of unsaturated monocarboxylic acids and which are normally referred to as dimer and trimer fatty acids.

Instead of the carboxylic acids, alkyl esters thereof may also be used. The unsaturated carboxylic acid alkyl esters preferably correspond to formula (II):

R²—[A²]—COOR³ (II)

in which A² is a mono- or polyunsaturated, linear or branched hydrocarbon radical containing 5 to 21 carbon atoms and R² is hydrogen or has the same meaning as R³ and R³ is a linear or branched alkyl group containing 1 to 18 carbon atoms. The alkyl esters are generally the ethyl, propyl, butyl, capryl and preferably methyl esters of the above-mentioned mono- and dicarboxylic acids. Similarly, instead of the alkyl esters, the polyol esters, especially the glycerol esters, may also be used. Preferred unsaturated carboxylic acid polyol esters are unsaturated mono-, di- and/or triglycerides which are normally natural oils with more or less large contents of unsaturated homologs in the mixture. Typical examples are palm oil, palm kernel oil, coconut oil and bovine tallow and, preferably, olive oil, sunflower oil, rapeseed oil, linseed oil, rice husk oil and the like. The unsaturated fatty compounds used typically have iodine values in the range from 10 to 300, preferably in the range from 50 to 200 and more particularly in the range from 70 to 125 which are reduced to values below 2, preferably below 1 and, more particularly, below 0.5. As mentioned at the beginning, the unsaturated fatty compounds may be used in the form of a mixture with their saturated homologs providing the iodine value is at least 10.

Transition Metal Catalysts

As mentioned at the beginning, suitable hydrogenation catalysts are transition metals among which nickel and especially palladium are particularly preferred. These metals are preferably applied to supports, silicon dioxide and especially active carbon being particularly suitable for this purpose. It has proved to be of particular advantage to use the Pd/active carbon catalysts described, for example, in EP 0632747 B1 (Henkel). Particulars of the production and use of such catalysts can be found in the disclosure of that document.

Hydrogenation

The hydrogenation of the unsaturated fatty compounds can be carried out in known manner, for example by arranging the catalyst in a fixed bed and contacting the educt and hydrogen with one another in co-current or counter-current. The hydrogenation temperatures are typically 150 or 220° C. and preferably 180 or 200° C. Although the process can of course also be carried out discontinuously, it is preferably carried out continuously because, in this way, a compressed hydrogen recycle gas can be used, eliminating the need for separate expansion and compression.

### EXAMPLES

General Procedure.

In a continuously operated pilot plant comprising a 25-liter fixed-bed reactor with a diameter of 60 mm and a length of 8,000 mm, unsaturated carboxylic acids were hydrogenated at temperatures of 180 to 200° C. and under pressures of 20 to 250 bar. The hydrogenation was carried out in the presence of the palladium/active carbon catalyst described in Example 1 of EP 0632747B1. An unsaturated C₁₈ dicarboxylic acid with an iodine value of 90 obtained by fermentation was used as the educt. The LHSV and iodine value (Wijs) were determined. The results are set out in Table 1. Examples 1 and 2 correspond to the invention, Examples C1 and C2 are intended for comparison.

TABLE 1
Hydrogenation of dicarboxylic acids

| Example | T [° C.] | p [bar] | H₂ expansion [Nm³/h] | LHSV [h⁻¹] | IV |
|---|---|---|---|---|---|
| C1 | 180 | 20 | 13 | 0.23 | 7.8 |
| C2 | 200 | 20 | 13 | 0.17 | 1.1 |
| 1 | 180 | 250 | 8 | 1.0 | 0.2 |
| 2 | 180 | 250 | 8 | 1.5 | 0.4 |

It can be seen that increasing the pressure from 20 to 250 bar results in a tenfold increase in the LHSV and hence in the reaction rate.

1-13. (canceled)

14. A process for the hydrogenation of an unsaturated fatty compound comprising contacting the unsaturated fatty compounds with hydrogen at a pressure of from 100 to 300 bar in the presence of transition metal catalyst to form a hydrogenation product having an iodine value of less than 2.

15. The process of claim 14 wherein the unsaturated fatty compound is an unsaturated carboxylic acid, an unsaturated carboxylic acid alkyl ester or an unsaturated carboxylic acid polyol ester.

16. The process of claim 15 wherein the unsaturated carboxylic acid is a compound of the formula (I):
R¹—[A¹]—COOH (I)
in which A¹ is a mono- or polyunsaturated, linear or branched hydrocarbon radical having from 5 to 21 carbon atoms and R¹ is hydrogen or a carboxyl group.

17. The process of claim 15 wherein the unsaturated carboxylic acid ester is a compound of the formula (II):
R²—[A²]—COOR³ (II)
wherein A² is a mono- or polyunsaturated, linear or branched hydrocarbon radical having from 5 to 21 carbon atoms and R² is hydrogen or is equal to R³ wherein R³ is a linear or branched alkyl group having from 1 to 18 carbon atoms.

18. The process of claim 14 wherein the unsaturated fatty compound is an unsaturated mono-, di- and/or triglyceride.

19. The process of claim 14 wherein the iodine value of the unsaturated fatty compound is from 10 to 300 are used.

20. The process of claim 14 wherein the unsaturated fatty compound is comprised of saturated homologs.

21. The process of claim 14 wherein the catalyst is nickel and/or palladium.

22. The process of claim 21 wherein the transition metal catalyst is deposited on a support.

23. The process of claim 22 wherein support is carbon or silicon dioxide.

24. The process of claim 14 wherein the catalyst is palladium on carbon.

25. The process of claim 14 wherein the process is carried out at a temperature of from 150 to 200° C.

26. The process of claim 14 wherein the process is carried out in a continuous fixed-bed reactor.
